(19) United States
(12) Patent Application Publication
(10) Pub. No.: US 2017/0035789 A1
(43) Pub. Date: Feb. 9, 2017

(54) Composition Of Dietary Supplements Based On Apoptosis that Support Optimal Health

(71) Applicants: Bill H. McAnalley, Grand Prairie, TX (US); Shayne A. McAnalley, Cedar Hill, TX (US); Erik H. Aguayo, Cedar Hill, TX (US)

(72) Inventors: Bill H. McAnalley, Grand Prairie, TX (US); Shayne A. McAnalley, Cedar Hill, TX (US); Erik H. Aguayo, Cedar Hill, TX (US)

(21) Appl. No.: 14/818,488

(22) Filed: Aug. 5, 2015

(51) Int. Cl. A61K 31/7004

(52) U.S. Cl. CPC A61K 31/7004; A23L 1/09; A23L 1/30; A23V 2002/00

(57) ABSTRACT

This invention clearly identifies the current agriculture-based diseases facing United States citizens such as, heart disease, stroke, cancer, diabetes and other degenerative diseases that did not exist in our hunter-gatherer ancestors. The United States government has established the Dietary Guidelines Advisory Committee (DGAC) to solve the problem. The DGAC's approach includes exercise, better agricultural based food choices plus vitamins-mineral supplements that are sex and age adjusted. We designed supplement compositions composed of unmodified dried fruits, herbs, including roots and tubers, many of which contain a lot of phytoestrogens. Currently, the medical profession has a bias against phytoestrogens, based on science that we believe is incorrectly interpreted. Today, women are afraid of getting breast cancer, and men are afraid of getting enlarged breasts from phytoestrogens. We will reinterpret the pre-clinical medical studies to alleviate these accepted concerns. Our supplement ingredients are chosen based on the different apoptosis pathways, which they stimulate, modulate or inhibit.

### RELATED APPLICATIONS

### References Cited

### U.S. Patent Documents

| | | |
|---|---|---|
| U.S. Pat. No. 5,149,528 | September 1992 | Maraganore et al. |
| U.S. Pat. No. 8,652,537 | February 2014 | Han et al. |
| U.S. Pat. No. 8,668,940 | March 2014 | Lee |
| U.S. Pat. No. 8,551,539 | October 2013 | Lee |
| U.S. Pat. No. 8,449,929 | May 2013 | Lee et al.. |
| U.S. Pat. No. 7,416,748 | August 2008 | Rangel |
| U.S. Pat. No. 8,652,537 | February 2014 | Han et al. |
| U.S. Pat. No. 7,618,657 | November 2009 | Fung et al. |
| U.S. Pat. No. 7,972,645 | July 2011 | Yang et al. |
| U.S. Pat. No. 7,838,050 | November 2010 | Randolph et al. |
| U.S. Pat. No. 7,758,902 | July 2010 | Randolph et al. |
| U.S. Pat. No. 8,491,943 | July 2013 | Ferguson et al. |
| 20140044817 A1 | February 2014 | Kim et al. |

### Foreign Patent Documents

| | | |
|---|---|---|
| CN102188592A | September 2011 | Batch |
| CN100534450C | September 2009 | Thin et a. |
| CN101161272A | April 2008 | Jizhong et al. |
| CN1874781B | November 2004 | Zhikai et al. |
| CN1227495A | September 1999 | McAnalley et al |

Ali, B. H., Al-Husseni, I., Beegam, S., Al-Shukaili, A., Nemmar, A., Schierling, S., Schupp, N. (2013). Effect of gum arabic on oxidative stress and inflammation in adenine-induced chronic renal failure in rats. PLoS One, 8(2), e55242. doi: 10.1371/journal.pone.0055242
Anderson, L. N., Cotterchio, M., Boucher, B. A., & Kreiger, N. (2013). Phytoestrogen intake from foods, during adolescence and adulthood, and risk of breast cancer by estrogen and progesterone receptor tumor subgroup among ontario women. Int J Cancer, 132(7), 1683-92. doi:10.1002/ijc.27788
Behr, M., Oehlmann, J., & Wagner, M. (2011). Estrogens in the daily diet: In vitro analysis indicates that estrogenic activity is omnipresent in foodstuff and infant formula. Food Chem Toxicol, 49(10), 2681-8. doi: 10.1016/j.fct.2011.07.039
Ben, X. M., U, J., Feng, Z. T., Shi, S. Y., Lu, Y. D., Chen, R., & Zhou, X. Y. (2008). Low level of galacto-oligosaccharide in infant formula stimulates growth of intestinal bifidobacteria and lactobacilli. World J Gastroenterol, 74(42), 6564-8.
Bilal, I., Chowdhury, A., Davidson, J., & Whitehead, S. (2014). Phytoestrogens and prevention of breast cancer: The contentious debate. World J Clin Oncol, 5(4), 705-12. doi:10.5306/wjco.v5.i4.705
Bogacz, A., Mikotajczak, P. [symbol] ., Mikotajczak, P. [symbol] ., Rakowska-Mrozikiewicz, B., Grze□kowiak, E., Wolski, H., Mrozikiewicz, P. M. (2014). The influence of soybean extract on the expression level of selected drug transporters, transcription factors and cytochrome P450 genes encoding phase I drug-metabolizing enzymes. Ginekol Pol, 85(5), 348-53.
Bowman, S. A., & Vinyard, B. T. (2004). Fast food consumption of U.S. Adults: Impact on energy and nutrient intakes and overweight status. J Am Coll Nutr, 23(2), 163-8.
Bröker, L. E., Kruyt, F. A., & Giaccone, G. (2005). Cell death independent of caspases: A review. Clinical Cancer Research: An Official Journal of the American Association for Cancer Research, 11(9), 3155-62. doi:10.1158/1078-0432.CCR-04-2223
Burton, R. M., & Westphal, U. (1972). Steroid hormone-binding proteins in blood plasma. Metabolism, 21(3), 253-276. doi:10.1016/0026-0495(72)90048-0
Cao, L. L., Xu, Y., Xu, S. L., Jin, M. M., & Shen, C. (2012). [Effects of snakegourd root polysaccharide on apoptosis of MCF-7 cells]. Zhejiang Da Xue Xue Bao Yi Xue Ban, 41(5), 527-34.
Carter, P., Achana, F., Troughton, J., Gray, L J., Khunti, K., & Davies, M. J. (2014). A mediterranean diet improves hba1c but not fasting, blood glucose compared to alternative dietary strategies: A network meta-analysis. J Hum Nutr Diet, 27(3), 280-97. doi: 10.1111/jhn. 12138
Chou, Y. C., Chang, M. Y., Wang, M. J., Hamod, T., Hung, C. H., Lee, H. T., . . . Chung, J. G. (2015). PEITC induces apoptosis of human brain glioblastoma GBM8401 cells through the extrinsic- and intrinsic-signaling pathways. Neurochem Int, 81, 32-40. doi: 10.1016/j.neuint.2015.01.001
Cordain, L, Eaton, S. B., Sebastian, A., Mann, N., Lindeberg, S., Watkins, B. A., . . . Brand-Miller, J. (2005). Origins and evolution of the western diet: Health implications for the 21st century. Am J Clin Nutr, 81(2), 341-54.
Dickens, L. S., Powley, I. R., Hughes, M. A., & MacFarlane, M. (2012). The ‘complexities’ of life and death: Death receptor signalling platforms. Exp Cell Res, 378(11), 1269-77. doi: 10.1016/j.yexcr.2012.04.005
Donkin, R. A. (1980). Manna: An historical geography (Vol. 17). Junk. Retrieved from Google Scholar.
dos Santos Silva, I., Mangtani, P., McCormack, V., Bhakta, D., McMichael, A. J., & Sevak, L. (2004). Phyto-oestrogen intake and breast cancer risk in south asian women in england: Findings from a population-based case-control study. Cancer Causes & Control: CCC, 15(8), 805-18. doi:10.1023/B:CAC0.0000043431.85706.d8
Eaton, S. B., & Konner, M. (1985). Paleolithic nutrition. A consideration of its nature and current implications. N Engl J Med, 312(5), 283-9. doi:10.1056/NEJ M198501313120505
Eaton, S. B., Eaton, S. B., & Konner, M. J. (1997). Paleolithic nutrition revisited: A twelve-year retrospective on its nature and implications. European Journal of Clinical Nutrition, 51(A), 207-16.
Evans, R. C., Fear, S., Ashby, D., Hackett, A., Williams, E., Van Der Vliet, M., . . . Rhodes, J. M. (2002). Diet and colorectal cancer: An investigation of the lectin/galactose hypothesis. Gastroenterology, 122(7), 1784-92.
Fulda, S. (2015). Targeting extrinsic apoptosis in cancer: Challenges and opportunities. Semin Cell Dev Biol, 39, 20-25. doi: 10.1016/j.semcdb.2015.01.006
Ganry, O. (2002). Phytoestrogen and breast cancer prevention. Eur J Cancer Prev, 11(6), 519-22.
Grein, A., da Silva, B. C., Wendel, C. F., Tischer, C. A., Sierakowski, M. R., Moura, A. B., Riegel-Vidotti, I. C. (2013). Structural characterization and emulsifying properties of polysaccharides of acacia meamsii de wild gum. Carbohydr Polym, 92(1), 312-20. doi: 10.1016/j.carbpol.2012.09.041
Hansen, K. L., Brustad, M., & Johnsen, K. (2015). Prevalence of self-reported stomach symptoms after consuming milk among indigenous sami and non-sami in northern- and mid-norway—the SAMINOR study. Int J Circumpolar Health, 74, 25762.
Hanson, L, Korotkova, M., & Telemo, E. (2003). Breast-feeding, infant formulas, and the immune system. Annals of Allergy, Asthma & Immunology, 90(6), 59-63. Retrieved from Google Scholar.
Hodgert Jury, H., Zacharewski, T. R., & Hammond, G. L (2000). Interactions between human plasma sex hormone-binding globulin and xenobiotic ligands. J Steroid Biochem Mol Biol, 75(2-3), 167-76.
Hughes, C., Davoodi-Semiromi, Y., Colee, J. C., Culpepper, T., Dahl, W. J., Mai, V., Langkamp-Henken, B. (2011). Galactooligosaccharide supplementation reduces stress-induced gastrointestinal dysfunction and days of cold or flu: A randomized, double-blind, controlled trial in healthy university students. Am J Clin Nutr, 93(6), 1305-11. doi:10.3945/ajcn.111.014126
Iwasaki, M., Inoue, M., Otani, T., Sasazuki, S., Kurahashi, N., Miura, T., Japan Public Health Center-based prospective study group. (2008). Plasma isoflavone level and subsequent risk of breast cancer among japanese women: A nested case-control study from the japan public health center-based prospective study group. J Clin Oncol, 26(10), 1677-83. doi:10.1200/JC0.2007.13.9964
Jiang, A. J., Jiang, G., Li, L T., & Zheng, J. N. (2015). Curcumin induces apoptosis through mitochondrial pathway and caspases activation in human melanoma cells. Mol Biol Rep, 42(1), 267-75. doi:10.1007/s11033-014-3769-2
Jönsson, T., Granfeldt, Y., Erlanson-Albertsson, C., Ahrén, B., & Lindeberg, S. (2010). A paleolithic diet is more satiating per calorie than a mediterranean-like diet in individuals with ischemic heart disease. Nutr Metab (Lond), 7, 85. doi:10.1186/1743-7075-7-85
Ju, Y., Xue, Y., Huang, J., Zhai, Q., & Wang, X. H. (2014). Antioxidant Chinese yam polysaccharides and its pro-proliferative effect on endometrial epithelial cells. Int J Biol Macromol, 66, 81-5. doi:10.1016/j.ijbiomac.2014.01.070
Jung, K. H., & Lee, K. H. (2015). Molecular imaging in the era of personalized medicine. J Pathol Transl Med, 49(1), 5-12. doi:10.4132/jptm.2014.10.24
Kelly, G. S. (1999). Larch arabinogalactan: Clinical relevance of a novel immune-enhancing polysaccharide. Altern Med Rev, 4(2), 96-103.
Kim, M. H., Kim, S. H., & Yang, W. M. (2014). Beneficial effects of astragaloside IV for hair loss via inhibition of fas/fas I-mediated apoptotic signaling. PLoS One, 9(3), e92984. doi: 10.1371/joumal.pone.0092984
Konner, M., & Eaton, S. B. (2010). Paleolithic nutrition: Twenty-five years later. Nutr Clin Pract, 25(6), 594-602. doi:10.1177/0884533610385702
Lai, J. N., Wu, C. T., Chen, P. C., Huang, C. S., Chow, S. N., & Wang, J. D. (2011). Increased risk for invasive breast cancer associated with hormonal therapy: A nation-wide random sample of 65,723 women followed from 1997 to 2008. PLoS One, 6(10), e25183. doi:10.1371/joumal.pone.0025183
Lartigue, L, Kushnareva, V., Seong, Y., Lin, H., Faustin, B., & Newmeyer, D. D. (2009). Caspase-independent mitochondrial cell death results from loss of respiration, not cytotoxic protein release. Mol Biol Cell, 20(23), 4871-84. doi:10.1091/mbc.E09-07-0649
de Lemos, M. L (2001). Effects of soy phytoestrogens genistein and daidzein on breast cancer growth. Ann Pharmacother, 35(9), 1118-21.
Li, Y., Wang, X., Cheng, S., Du, J., Deng, Z., Zhang, Y., . . . Ling, C. (2015). Diosgenin induces G2/M cell cycle arrest and apoptosis in human hepatocellular carcinoma cells. Oncol Rep, 33(2), 693-8. doi:10.3892/or.2014.3629
Limpeanchob, N., Jaipan, S., Rattanakaruna, S., Phrompittayarat, W., & Ingkaninan, K. (2008). Neuroprotective effect of bacopa monnieri on beta-amyloid-induced cell death in primary cortical culture. Journal of Ethnopharmacology, 720(1), 112-7. doi: 10.1016/j.jep.2008.07.039
Lindeberg, S., & Lundh, B. (1993). Apparent absence of stroke and ischaemic heart disease in a traditional melanesian island: A clinical study in kitava. J Intern Med, 233(3), 269-75.
Liu, L, Bestel, S., Shi, J., Song, Y., & Chen, X. (2013). Paleolithic human exploitation of plant foods during the last glacial maximum in north china. Proc Natl Acad Sci USA, 770(14), 5380-5. doi:10.1073/pnas.1217864110
London, D. S., & Beezhold, B. (2015). A phytochemical-rich diet may explain the absence of age-related decline in visual acuity of amazonian hunter-gatherers in ecuador. Nutr Res, 35(2), 107-17. doi:10.1016/j.nutres.2014.12.007
Lopez, J., & Tait, S. W. (2015). Mitochondrial apoptosis: Killing cancer using the enemy within. Br J Cancer, 112(6), 957-62. doi:10.1038/bjc.2015.85
Magee, P. J., & Rowland, I. (2012). Soy products in the management of breast cancer. Curr Opin Clin Nutr Metab Care, 75(6), 586-91. doi:10.1097/MCQ.0b013e328359156f
Martin, P. M., Horwitz, K. B., Ryan, D. S., & McGuire, W. L. (1978). Phytoestrogen interaction with estrogen receptors in human breast cancer cells. Endocrinology, 703(5), 1860-7. doi: 10.1210/endo-103-5-1860
Messina, M. (2014). Soy foods, isoflavones, and the health of postmenopausal women. Am J Clin Nutr, 100 Suppl 1, 423S-30S. doi:10.3945/ajcn.113.071464
Ni, L., Zhu, X., Gong, C., Luo, Y., Wang, L., Zhou, W Li, Y. (2015). Trichosanthes kirilowii fruits inhibit non-small cell lung cancer cell growth through mitotic cell-cycle arrest. Am J Chin Med, 43(2), 349-64. doi: 10.1142/S0192415X15500238
Pan, K. Z., Un, Y. J., Fu, Z. J., Zhou, K. J., Cai, Z. P., Chen, Z. W., Ma, X. Q. (1987). The three-dimensional structure of trichosanthin molecule. Sci Sin B, 30(4), 386-94.
Panossian, A., & Wagner, H. (2005). Stimulating effect of adaptogens: An overview with particular reference to their efficacy following single dose administration. Phytother Res, 79(10), 819-38. doi:10.1002/ptr. 1751
Penolazzi, L, Lampronti, I., Borgatti, M., Khan, M. T., Zennaro, M., Piva, R., & Gambari, R. (2008). Induction of apoptosis of human primary osteoclasts treated with extracts from the medicinal plant emblica officinalis. BMC Complement Mem Med, 8, 59. doi:10.1186/1472-6882-8-59
Peter, M. E., Hadji, A., Murmann, A. E., Brockway, S., Putzbach, W., Pattanayak, A., & Ceppi, P. (2015). The role of CD95 and CD95 ligand in cancer. Cell Death Differ, 22(4), 549-59. doi:10.1038/cdd.2015.3
Pflaum, J., Schlosser, S., & Müller, M. (2014). P53 family and cellular stress responses in cancer. Front Oncol, 4, 285. doi:10.3389/fonc.2014.00285
Piyabhan, P., & Wetchateng, T. (2014). Neuroprotective effects of bacopa monnieri (brahmi) on novel object recognition and NMDAR1 immunodensity in the prefrontal cortex, striatum and hippocampus of sub-chronic phencyclidine rat model of schizophrenia. J Med Assoc Thai, 97 Suppl 8, S50-5.
Powers, C. N., & Setzer, W. N. (2015). A molecular docking study of phytochemical estrogen mimics from dietary herbal supplements. In Silico Pharmacology, 3, 4. doi: 10.1186/S40203-015-0008-z
Prakash, J., Chouhan, S., Yadav, S. K., Westfall, S., Rai, S. N., & Singh, S. P. (2014). Withania somnifera alleviates parkinsonian phenotypes by inhibiting apoptotic pathways in dopaminergic neurons. Neurochemical Research, 39(12), 2527-2536. Retrieved from Google Scholar.
Prietsch, R. F., Monte, L G., Da Silva, F. A., Beira, F. T., Del Pino, F. A. B., Campos, V. F., . . . Gamaro, G. D. (2014). Genistein induces apoptosis and autophagy in human breast MCF-7 cells by modulating the expression of proapoptotic factors and oxidative stress enzymes. Molecular and Cellular Biochemistry, 390(1-2), 235-242. Retrieved from Google Scholar.
Reid, J. M., Goetz, M. P., Buhrow, S. A., Walden, C., Safgren, S. L., Kuffel, M. J., . . . Ames, M. M. (2014). Pharmacokinetics of endoxifen and tamoxifen in female mice: Implications for comparative in vivo activity studies. Cancer Chemother Pharmacol, 74(6), 1271-8. doi:10.1007/s00280-014-2605-7
Seo, C.-S., Kim, T.-W., Kim, Y.-J., Park, S.-R., Ha, H., Shin, H.-K., & Jung, J.-Y. (2015). Trichosanthes kirilowii ameliorates cisplatin-induced nephrotoxicity in both in vitro and in vivo. Natural Product Research, 29(6), 554-557. Retrieved from Google Scholar.
Shalini, S., Dorstyn, L., Dawar, S., & Kumar, S. (2014). Old, new and emerging functions of caspases. Cell Death and Differentiation, 22(4), 526-539. doi:10.1038/cdd.2014.216
Slattery, M. L, Sorenson, A. W., Mahoney, A. W., French, T. K., Kritchevsky, D., & Street, J. C. (1988). Diet and colon cancer: Assessment of risk by fiber type and food source. Journal of the National Cancer Institute, 80(18), 1474-1480. doi:10.1093/jnci/80.18.1474
Sun, B., Geng, S., Huang, X., Zhu, J., Liu, S., Zhang, Y Wang, J. (2011). Coleusin factor exerts cytotoxic activity by inducing G0/G1 cell cycle arrest and apoptosis in human gastric cancer BGC-823 cells. Cancer Lett, 301(1), 95-105. doi: 10.1016/j.canlet.2010.10.010
Swagerty, D. L, Walling, A. D., & Klein, R. M. (2002). Lactose intolerance. Am Fam Physician, 65(9), 1845-50.
Tait, S. W., & Green, D. R. (2010). Mitochondria and cell death: Outer membrane permeabilization and beyond. Nat Rev Mol Cell Biol, 11(9), 621-32. doi:10.1038/nrm2952
Tait, S. W., Ichim, G., & Green, D. R. (2014). Die another way—non-apoptotic mechanisms of cell death. J Cell Sci, 127(Pt 10), 2135-44. doi:10.1242/jcs.093575
Taylor, R. C., Cullen, S. P., & Martin, S. J. (2008). Apoptosis: Controlled demolition at the cellular level. Nat Rev Mol Cell Biol, 9(3), 231-41. doi:10.1038/nrm2312
Tousen, Y., Uehara, M., Abe, F., Kimira, Y., & Ishimi, Y. (2013). Effects of short-term fructooligosaccharide intake on equol production in japanese postmenopausal women consuming soy isoflavone supplements: A pilot study. Nutr J, 12, 127. doi:10.1186/1475-2891-12-127
Williams, M. M., & Cook, R. S. (2015). Bcl-2 family proteins in breast development and cancer: Could mcl-1 targeting overcome therapeutic resistance? Oncotarget, 6(6), 3519-30.
Williams, R., Munch, G., Gyengesi, E., & Bennett, L. (2014). Bacopa monnieri (L.) Exerts anti-inflammatory effects on cells of the innate immune system in vitro. Food & Function, 5(3), 517-520. Retrieved from Google Scholar.
Yang, G., Yang, L., Zhuang, Y., Qian, X., & Shen, Y. (2014). Ganoderma lucidum polysaccharide exerts anti-tumor activity via MAPK pathways in HL-60 acute leukemia cells. Journal of Receptors and Signal Transduction, (0), 1-8. Retrieved from Google Scholar.
Yeung, H. W., & Li, W. W. (1987). Beta-trichosanthin: A new abortifacient protein from the Chinese drug, wangua, trichosanthes cucumeroides. International Journal of Peptide and Protein Research, 29(3), 289-92.
Yukawa, H., Ishikawa, S., Kawanishi, T., Tamesada, M., & Tomi, H. (2012). Direct cytotoxicity of lentinula edodes mycelia extract on human hepatocellular carcinoma cell line. Biol Pharm Bull, 35(7), 1014-21.
Zeng, W., Wang, X., Xu, P., Liu, G., Eden, H. S., & Chen, X. (2015). Molecular imaging of apoptosis: From micro to macro. Theranostics, 5(6), 559-82. doi:10.7150/thno.11548

### FIELD OF THE INVENTION

This invention is in the field of dietary supplements composed of pre-agrarian food or plant ingredients that modulate apoptosis pathways at different intra- and extra-cellular sites to achieve the desired health effects.

### BACKGROUND AND DESCRIPTION OF THE PRIOR ART

The government recognizes the problem. Their solution started with the Recommended Daily Allowances (RDA), established by earlier U.S. governmental agencies during World War II for the military, civilian, and overseas populations needing food relief. They were approved in 1941, and used until 1997, when the RDA became part of a broader, more detailed, dietary guideline called the Dietary Reference Intake, (RDI) which is updated every five to ten years.

The 2015 Dietary Guidelines Advisory Committee (DGAC) was established jointly by the secretaries of the U.S. Department of Health and Human Services (HHS) and the U.S. Department of Agriculture (USDA). The Committee was charged with examining the Dietary Guidelines for Americans, 2010 and to determine topics for which new scientific nutritional evidence was available for all Americans, two years and older.

Estimated Average Requirement, or Adequate Intake Levels, and the Tolerable Upper Intake Levels are currently set by the Institute of Medicine, (IOM). They were determined through the average intake, (or the excessive intake), by healthy members grouped by sex and age. The IOM has established both an Average Requirement and the Upper Intake Level for vitamins and minerals.

The 2015 DGAC's work was guided by two fundamental realities. First, about half of all American adults—117 million individuals—have one or more preventable chronic diseases, and second, about two-thirds of U.S. adults—nearly 155 million individuals—are overweight or obese. These conditions have been highly prevalent in the general population for more than two decades. Poor dietary patterns, overconsumption of calories, and physical inactivity, directly contribute to these disorders.

The DGAC have hoped that positive changes in individual diet and physical activity behaviors would have substantially improved health outcomes. This has not been the case.

The DGAC found that several nutrients are still under-consumed relative to the Estimated Average Requirement or Adequate Intake Levels set by the Institute of Medicine (IOM), and the committee characterized these as short-fail nutrients: vitamin A, vitamin D, vitamin E, vitamin C, folate, calcium, magnesium, fiber, and potassium. For adolescent and premenopausal females, iron is also a shortfall nutrient. Of these shortfall nutrients, calcium, vitamin D, fiber, and potassium also are classified as nutrients of public health concern, due to under consumption linked in the scientific literature to adverse health outcomes. Iron is included as a shortfall nutrient for adolescent females, and adult females, who are premenopausal due to the increased risk of iron-deficiency in these groups.

The DGAC also found that two nutrients—sodium and saturated fat—are over-consumed by the U.S. population relative to the Tolerable Upper Intake Level set by the IOM and this over-consumption poses health risks.

The sedentary practices of most modern jobs require a fraction of the calories that our ancestors used when they spent all day farming, gardening, hunting, walking and occasionally running. Therefore, our diet requires significantly less calories than our ancestors' diet to maintain a healthy weight. An overwhelming exercise deficiency started after World War II, when people's energy requirements were reduced much faster than their energy consumption, resulting in weight problems. Rapid industrialization has created new jobs requiring less energy per hour. The approximate energy in calories required per hour for different jobs and the activities of an average person, weighing between 150 and 155 pounds, is listed below:

| | |
|---|---|
| Desk job | 106 |
| Retail Job | 162 |
| Childcare Worker | 211 |
| Walking the dog | 246 |
| Gym Teacher | 282 |
| Walking on grass | 320 |
| Gardening | 352 |
| Hunting, general | 352 |
| Hiking, cross-country | 422 |
| Walking on packed snow | 450 |
| Backpacking | 493 |
| Basketball | 563 |
| Baling hay | 563 |
| Swimming at a fast pace | 665 |
| Walking in snow powder | 700 |
| Bicycling very fast | 844 |
| Running, 6 min miles | 1126 |

Can more exercise solve the problem? We can start by comparing the energy required to work at a desk, which is 106 calories per hour, to a more strenuous activity, like gardening, which burns 352 calories per hour. In a typical eight-hour day, the desk job requires 848 calories, where gardening requires 2,816 calories, burning 1,968 more calories per day. The desk-worker would have to swim three hours a day, or walk their dog eight hours a day to burn the extra calories. This extends the sedentary worker's day to either an 11 or 16-hour day, depending upon their exercise choice. By the time a person travels to their desk job, works and goes home, most people are both mentally and physically too tired to burn the calories. It is unlikely that more exercise atone will make much of a difference.

Problems with our modern diet: When agriculture first started about 10,000 years ago, it did not bring about an increase in health, but rather the opposite. The first farmers had a shorter lifespan, they were significantly smaller, and were generally less healthy than their hunter-gatherer predecessors. Infant mortality, infectious diseases, bone mineral disorders, dental caries and iron deficiency anemia were greatly increased (Eaton & Konner, 1985; Eaton, Eaton, & Konner, 1997; Konner & Eaton, 2010).

Today we know that agriculture-based diets have contributed to many problems: Heart disease, stroke, cancer, diabetes and other degenerative diseases did not exist in our hunter-gatherer ancestors' time (Cordain et al., 2005). As these new health problems appeared, drugs have been developed to manage the symptoms, but not necessarily the causes. The drugs have brought with them a new set of health problems; i.e. toxicities and side effects.

Our food requirements were developed over millions of years by hunter-gatherer diets, creating biochemical requirements for food that existed before agriculture. Genetically, our bodies are virtually the same as they were at the end of the Paleolithic era, 20,000 years ago. The best diet for human health and well-being should resemble our Paleolithic ancestral diet (Eaton & Konner, 1985; Eaton et al., 1997; Konner & Eaton, 2010).

For example Staffan Lindeberg, a Swedish medical doctor and scientist, conducted several scientific surveys on the non-westernized population at the Kitava Island in the Solomon Sea. These surveys, collectively referred to as the Kitava Study, found the population currently living on the hunter-gatherer diet do not suffer from stroke, ischemic heart disease, diabetes, obesity, or hypertension (Lindeberg & Lundh, 1993). These same findings have been observed in other hunter-gatherers all over the world (London & Beezhold, 2015).

Perhaps the best answer to agriculturally based health challenges is to analyze the differences in the hunter-gatherer diet and our modern diet.

The hunter gatherer diet: The Paleolithic era extends from 2.5 million years ago to 10,000 years ago. The hunter-gatherer diet, also referred to as the “caveman diet” or the “paleolithic diet”, consists mainly of fish, range-fed meats, vegetables, fruits, roots, tubers and nuts. They consumed many different fruits and vegetables, from many different plants in each area that they lived. The caveman diet mainly excluded grains, legumes, dairy products, refined sugar, salt, and processed vegetable oils (Eaton & Konner, 1985; Eaton et al., 1997; Konner & Eaton, 2010).

The hunter-gatherer diet had fewer calories per gram than the average US diet. Most fruits and berries contain 0.4 to 0.8 calories per gram; vegetables usually have even less. Modern foods, like hamburgers and sandwiches, have 2.4 to 2.8 calories per gram; cookies and chocolate bars commonly exceed 4 calories per gram. High-energy-dense diets can contribute to health problems in the following three areas (Cordain et al., 2005; Eaton & Konner, 1985; Eaton et al., 1997; Konner & Eaton, 2010).

First, substantial evidence demonstrates that people who consume high-energy-dense diets are prone to overeating, because they consume a lot more calories before their stomach stretches enough to fell them that they are full. Consequently, they are at a greater risk of obesity. Conversely, diets with lower caloric density provide a feeling of fullness with fewer calories.

Second, not only did the caveman consume fewer calories per bite, he ate more fruits and vegetables, which leads to a much higher fiber intake. Hunter-gatherer diets include uncultivated, high-fibrous fruit and vegetables. Some caveman diets contained more than 100 grams of fiber per day, which is dramatically higher than the current average United States intake of 115 grams per day.

Third, the cave man diet had more vitamins minerals and antioxidants than most of today's diets. Fruits, vegetables, seafood, meat and organ meats were the staples of the hunter-gatherer diet. These foods are more micronutrient-dense than today's grains, vegetable oils, and dairy products. Consequently, the vitamin and mineral content of the caveman diet was usually many times the current Recommended Daily Allowance.

Lactose Intolerance: Recent evidence indicates that up to 75% of the world's population is lactose intolerant to some extent That is, three quarters of all people have difficulty digesting the milk sugar called lactose (Swagerty, Walling, & Klein, 2002).

Domestication of dairy animals, and consumption of milk, started about 9,000 years ago in western Asia. DNA samples, from people who lived in Europe 9,000 years ago, revealed that they were lactose-intolerant at that period in time. Before the domestication of dairy animals, all human adults were lactose intolerant based on DNA from ancient burial sites.

Lactose intolerance is a deficiency of lactase, the enzyme in your small intestine that splits lactose into glucose and galactose. Unlike lactose, glucose and galactose can be absorbed into your blood. The excess lactose is digested by bacteria in the intestines, where it can cause gas, cramping, and diarrhea.

In the United States and Europe, the prevalence of lactose intolerance is 7% to 20% in Caucasian adults, (the lowest being in northern Europeans), and it is as high as 80% to 95% among Native Americans, 65% to 75% among Africans and African Americans, and 50% in Hispanics. Lactose intolerance develops between ages five and seven in the United States. No one develops lactose intolerance before age two (Hansen, Brustad, & Johnsen, 2015). Consumption of dairy started ten thousand years ago, yet most of the world is still lactose-intolerant demonstrating how long it takes mankind to genetically adapt to new foods.

What are Leptins? Leptins are hormones that help regulate energy intake and energy expenditure. They also control appetite and metabolism. The amount of circulating leptin is directly proportional to the total amount of fat in the body. Leptins are produced by fat tissue, ovaries, mammary cells, muscle, bone marrow, liver, and gastric cells in the stomach.

Leptins act on brain receptors in the hypothalamus, where they inhibit appetite by counteracting the effects of two powerful appetite stimulants, neuropeptide Y and anandamide. The absence of leptin, or it's receptor, leads to uncontrolled food intake and obesity.

Tommy Jönsson used leptin concentrations to demonstrate that the Paleolithic diet was more satisfying per calorie than the Mediterranean diet (Jonsson, Granfeldt, Erlanson-Albertsson, Ahrén, & Lindeberg, 2010). In other words, the Paleolithic dietary group felt as full as the Mediterranean dietary group, but with fewer calories. This helps to explain why people crave more food even after they have consumed more calories than needed to maintain their weight.

Advocates of the hunter-gatherer diet believe that fast food, and the consumption of modern agricultural food, is responsible for the current epidemic levels of obesity, type-2 diabetes, osteoporosis, cardiovascular disease, high blood pressure, and cancer (Bowman & Vinyard, 2004; Konner & Eaton, 2010). More than 70% of the total daily energy, consumed by all people in the United States, comes from dairy products, cereals, refined sugars, processed oils and alcoholic beverages. These foods did not even exist in the time of the hunter-gatherer.

Many controlled clinical trials have shown that the hunter-gatherer diet is superior to other modern diets, like the Mediterranean diet, which includes high olive oil consumption, high consumption of legumes, unrefined cereals, fruits and vegetables (Carter et al., 2014).

Currently, oncologists still advise women to not take phytoestrogens, because they could increase the risk of breast and ovarian cancer. The concern comes from two sources. First, hormone replacements, like estrogen, or estrogen plus progesterone, increase the risk of breast cancer in Western countries. Recently, a similar randomized study, with the same hormone therapy, was conducted in a nation-wide Taiwanese study using 65,723 Chinese women. The hormone therapy increased the risk of invasive breast cancer in the Taiwanese women. There was no difference in the risk of estrogen-induced invasive breast cancer in women from Asian or Western countries, proving no effect caused by genetic difference between the groups of women (Lai et al., 2011).

Second, estrogen and phytoestrogens are believed to represent a similar cancer risk; based on preclinical evaluations. Oncologists recommended that women should be aware of the potential cancer risk from phytoestrogens. The oncologists' concern has caused women to shy away from phytoestrogens, and for men to be afraid of getting larger breasts.

The interest in phytoestrogens started when epidemiological studies found that Asian women, who consume high dietary concentrations of soy products, have a lower incidence of breast cancer (Bilal, Chowdhury, Davidson, & Whitehead, 2014).

This prompted scientists to conduct preclinical studies with phytoestrogens. To their surprise low concentrations of phytoestrogens stimulated breast-cancer cell growth in tissue culture and in athymic mice in-vivo studies. Also, it inhibited the anti-tumor effect of tamoxifen, but higher concentrations of the same phytoestrogens inhibited tumor growth and enhanced the effect of tamoxifen. Until the medical profession could explain the difference, they cautioned women about the possible increased risk of breast cancer. (Ganry 2002; de Lemos 2001; Martin, Horwitz, Ryan, & McGuire, 1978).

Our interpretation of the problem follows: First, the intake of traditional soy-based foods is high in Japan, and the mean total intake of isoflavones is estimated to be between 19.4 and 33.6 mg per day, according to a National Nutritional Survey in Japan.

In Western populations, the consumption of isoflavones from traditional soy foods is substantially lower between 0.5 and 3 mg (Tousen, Uehara, Abe, Kimira, & Ishimi, 2013). Another scientific group found phytoestrogens in all food analyzed and reported them to be omnipresent, not limited to soy-based food. (Behr, Oehlmann, & Wagner, 2011) Investigators should have recognized that phytoestrogens are in all foods. The average Western vegetarian consumes 1-3 mg per day of isoflavones, while Asians consume about 50 mg per day. (Bilal et al., 2014)

Phytoestrogens are in all fruits, vegetables, nuts, fish, other meats and surface drinking wafer supplies. In other words, phytoestrogens are in everything we consume. Western populations consume a low daily amount of the phytoestrogens that are shown to increase risk of breast cancer, based on the preclinical tissue culture, and athymic mice studies mentioned above. This suggests a conclusion that the only safe recommendation for people, who want to avoid breast-cancer risk caused by a diet tow in concentrations of phytoestrogens, is to stop eating all together, or increase their consumption to the proven safer amounts consumed by Asians.

These preclinical findings have been repeated many times, and we believe they are accurate. However, we can explain how low concentrations of phytoestrogens stimulated breast-cancer cell growth in tissue culture, and in athymic mice in-vivo studies, and inhibited the anti-tumor effect of tamoxifen.

To understand these preclinical findings, a review of the clinical chemistry of sex hormones is needed. Estrogen represents an entire class of related hormones including estriol, estradiol and estrone. The placenta makes estriol during pregnancy. Estradiol is the primary sex hormone of childbearing women. It is made in developing ovarian follicles, and is responsible for female characteristics and sexual functions. Estrone is the primary estrogen made after menopause.

There are two types of serum proteins form dissociable complexes with circulating sex hormones. Albumin is the most abundant plasma protein, and binds sex hormones, with a very low-binding affinity, predominantly by hydrophobic binding. The highly specific sex hormone-binding globulin (SHBG), also called the sex steroid-binding globulin (SSBG), occurs in low concentrations, but binds sex hormones with a very high-binding affinity, several orders of magnitude higher than albumin. The relative binding affinity of various sex hormones for SHBG is dihydrotestosterone>testosterone>androstenediol>estradiol>estrone (Hodgert Jury, Zacharewski, & Hammond, 2000).

Albumin-bound sex hormones constitute an inactive pool, protected from metabolic and chemical alterations, providing reserve hormones through reversible dissociation. Albumin also buffers against sudden changes in active hormone concentrations (Burton & Westphal, 1972).

All human tissue culture mediums contain human and/or animal serums, which automatically come with the sex-hormone bound to the albumin. The preclinical findings can be explained using the relative binding affinities (RBAs) of sex hormones to albumin by competitive displacement. When phytoestrogens are added at tow concentrations to the tissue culture, they competitively displace estrogen from albumin's weak binding sites. The newly freed estrogen will stimulate the estrogen (+) cancer cells.

As more and more phytoestrogen is added to the tissue culture medium, it will begin to competitively displace the newly bound phytoestrogen, which results in a net no-effect. Higher concentrations of phytoestrogen can successfully compete with estrogen for the estrogen receptor, and competitively inhibit the estrogen effect. The same is true for athymic mice grafted with human estrogen (+) cancer (Hodgert Jury et al., 2000).

How did higher concentrations of the same phytoestrogens enhance the effect of tamoxifen in the athymic mice grafted with human estrogen (+) cancer? Tamoxifen competes with estrogen for the estrogen receptor just like phytoestrogens. Tamoxifen is a weak anti-estrogen and has a low binding affinity for the estrogen receptor, but it can be metabolized in the liver by the cytochrome p-450 enzyme system, into 4-Hydroxytamoxifen (4HT) which has 100-fold greater binding affinity for the estrogen receptor, and a 30-fold to 100-fold greater potency in suppressing estrogen-dependent cell proliferation compared with un-metabolozed Tamoxifen. This is why estrogen (+) breast cancer patients have less of a chance of surviving if they have low cytochrome p-450 activity (Reid et al., 2014).

Recently, the soybean phytoestrogens, genistein and daidzein, have been shown to significantly increase cytochrome p-450 activity when compared with the control group (Bogacz et al., 2014). As in humans, the athymic mouse's cytochrome p-450 liver enzyme system is enhanced by the higher concentrations phytoestrogens which metabolizes more Tamoxifen info (4HT) that has 100-fold greater binding affinity for the estrogen receptor, and blocks estrogen's ability to bind the estrogen (+) cancer tumor receptors and stimulate its growth.

Induction of apoptosis by phytoestrogens provides another reason for higher concentrations of phytoestrogens killing human estrogen (+) cancer. Genistein, a soy isoflavone, has been reported to have a chemo-preventive and chemotherapeutic potential in multiple tumor types, including MCF-7 human estrogen (+) cancer cells. Genistein increases the proapoptotic BAX/Bcl-2 ratio by a factor of three, and down-regulates the protein preventing apoptosis, called survivin, by a factor of 20, resulting in apoptosis and cell death (Prietsch et al., 2014). This provides an additional support, for use of higher concentrations of phytoestrogens, stopping the growth of estrogen (+) cancer cells.

The epidemiology studies that follow should no longer be in conflict with the preclinical studies.

A total of 240 South Asian breast cancer cases living in England and 477 age-matched population-based controls were recruited into this study. Conditional logistic regression models were used to estimate the effect of phytoestrogen intake on breast cancer risk. Their findings were consistent with the possibility that high phytoestrogen intake may protect against breast cancer, but further research is required to confirm this hypothesis, (dos Santos Silva et al., 2004)

A total of 24,226 women ages 40 to 69 years in the Japan Public Health Center-based prospective study who responded to the baseline questionnaire and provided blood in 1980 to 1995 were observed up to December 2002. This nested, case-control study found an inverse association between plasma genistein and the risk of breast cancer in Japan. (Iwasaki et al., 2008)

Meta-analyses of epidemiological studies of soy consumption and breast cancer risk have demonstrated modest protective effects, usually attributed to isoflavones. Importantly, soy does not appear to interfere with tamoxifen or anastrozole therapy. Recent research suggests that women who are at increased risk of breast cancer due to polymorphisms in genes associated with the disease may especially benefit from high soy isoflavone intake. (Magee & Rowland, 2012)

Adolescent phytoestrogen intake was associated with reduced postmenopausal breast cancer, particularly for ER+PR+ tumor subgroup. (Anderson, Cotterchio, Boucher, & Kreiger, 2013)

Although concerns have been raised that soy food consumption may be harmful to breast cancer patients, an analysis in 9514 breast cancer survivors who were followed for 7.4 years found that higher post diagnosis soy intake was associated with a significant 25% reduction in tumor recurrence. In summary, the clinical and epidemiological data indicate that adding soy foods to the diet can contribute to the health of postmenopausal women. (Messina 2014)

The summation of these studies prove the incidents of breast cancer have been greatly reduced in populations consuming higher amounts of phytoestrogens, in both the Asian and Western populations, after controlling for cultural dietary differences.

The explanation provided above has not been obvious to our scientific peers. Today, oncologists still advise women not to take phytoestrogens. Over the last two decades, more than 1200 PubMed articles have been written about phytoestrogen and breast cancer, with conflicting recommendations. The scientific community has not linked the evidence provided in the studies above.

They are still having difficulties reconciling the epidemiological studies with the preclinical studies. For example, in a recent publication, “Phytoestrogens and prevention of breast cancer: The contentious debate,” authors verify that the scientific community is still biased against phytoestrogens (Bilal et al., 2014).

A 2015 study published by the Chemistry department at the University of Alabama, authors identified 568 phytochemicals in 17 of the most popular herbal supplements sold in the United States. This study has revealed that almost all popular herbal supplements contain phytoestrogen components, which bind to the human estrogen receptor, and may cause unwanted side effects related to estrogenic activity (Powers & Setzer, 2015).

The scientific community still considers phytoestrogens a health problem; this is opposed to our belief that their deficiency is a major cause of a many current agriculture-based diseases.

Instead of resolving the perceived problems, the scientific community is busy developing new classes off drugs that are highly specific derivatives of phytoestrogens, with a single drug indication.

The World Journal of Clinical Oncology cites that phytoestrogens provide multiple targets on breast cancer cells and their ability to modulate epigenetic events associated with breast cancer, and this prevention may lead to new, non-toxic therapeutic approaches through development of highly specific and long-acting analogues of phytoestrogens. (Bilal et al., 2014)

Apoptosis, or programmed cell death, is involved in numerous human conditions including neurodegenerative diseases, ischemic damage, autoimmune disorders and many types of cancer, and it is often confused with other types of cell death. Therefore, strategies that enable visualized detection of apoptosis would be of enormous benefit in the clinic for diagnosis, patient management, and the development of new therapies. (Zeng et al., 2015)

Since apoptosis is typically disrupted in human cancers, therapeutic targeting of apoptosis represents a promising avenue for the development of novel therapeutic approaches. This strategy is particularly relevant, because many currently used cancer therapies use apoptosis signaling pathways to exert their anti-tumor activities. A better understanding of these signaling networks and their deregulation of human cancers is anticipated to open new perspectives for the development of apoptosis-targeted therapies for the treatment of cancer. (Fulda 2015)

Apoptosis imaging is expected to make major contributions to personalized medicine by allowing earlier diagnosis and predicting treatment response. The technique is also making a huge impact on pharmaceutical development by optimizing preclinical and clinical tests for new drug candidates. This review will describe the basic principles of molecular imaging and will briefly touch on three examples (from an immense list of new techniques) that may contribute to personalized medicine: receptor imaging, angiogenesis imaging, and apoptosis imaging. (Jung & Lee, 2015)

Apoptosis screens are being used to discover new drugs, not to for a rational supplement design. These derivatives, and their analogues, will be considered a New Chemical Entity (NCE) by the FDA, and will require a New Drug Application (NDA) before they can enter the market. As of April 2015, there were 1446 PubMed articles on phytoestrogen derivatives, including 56 PubMed articles on phytoestrogen analogues. Most new drugs are derivatives of naturally occurring molecules from plants, fungus and other life forms. Using molecular drug design methods, drug companies are able to synthesize NCEs with more potency and efficiency, but NCEs usually have unexpected and unintended clinical problems. On the positive side, NCEs are easy to patent.

NCEs go through preclinical pharmaceutical screening taking two to five years before starting human clinical trials. If the preclinical studies are found to be satisfactory, the drug companies and FDA jointly develop and agree on the clinical studies for each phase. This process can take more than fen years.

Drug companies and the government are spending billions to develop a better understanding of the apoptosis signaling pathways and to develop NCEs. They plan to open new perspectives for the development of apoptosis-targeted therapies for neurodegenerative diseases, ischemic damages, autoimmune disorders and many types of cancer.

In summary, the medical professionals, drug companies, as well as the NIH and the FDA, are excited about the potential new apoptosis-based drug approaches for the agriculture-based diseases.

Cells die either by necrosis or apoptis: Cells that die, as a result of injury, swell and spill their contents all over their neighbors. This process is called cell necrosis, and usually results in a damaging inflammatory response. By contrast, a cell undergoing apoptosis dies without damaging its neighbors. This cell shrinks, condenses, and then collapses. Next, the nuclear membrane surrounding the nucleus disassembles. Subsequently, the nuclear DNA breaks up into fragments. The cell surface structure is altered to cause it to be rapidly phagocytosed, either by a neighboring cell, or by a macrophage. Every minute of life, millions of cells in our bodies undergo this naturally regulated form of cell death, called apoptosis. Apoptosis is the natural and healthy end to damaged or abnormal cells (Lopez & Tait, 2015).

The apoptis pathways: Apoptosis has been recognized, with the advent of more sensitive biochemical assays in the mid-1980s, through evidence demonstrating mitochondria's central role in apoptotic cell death. Caspase protease activity is essential for apoptosis. Once activated, caspase enzymes cleave hundreds of different proteins, leading to rapid cell death with distinctive biochemical and morphological hallmarks (Taylor, Cullen, & Martin, 2008).

In general terms, caspase activity can be initiated extrinsically to the cell by the cell-surface death-receptors, (reviewed in (Dickens, Powley, Hughes, & MacFarlane, 2012)) or intrinsically to the cell, by mitochondrial pathways of apoptosis (Tait & Green, 2010).

The defining event for apoptosis is the ‘Mitochondrial Outer Membrane Permeabilization’ (MOMP). Following MOMP, the mitochondrial proteins in the inter-membrane space, notably cytochrome c, is released into the cytosol, whereupon it activates the caspases. Cytochrome c has a normal function; shuttling electrons between complexes III and IV of the electron transport chain. However, once released from mitochondria, cytochrome c adopts a lethal function essential for caspase activation. Once in the cytosol, cytochrome c binds to the adaptor molecule, called ‘Apoptotic Protease Activating Factor-1’ (APAF-1); leading to extensive conformational changes in APAF-1, causing if to oligomerise and form a heptameric structure called an apoptosome.

The apoptosome signals, recruits, and activates pro-caspase-9. This protein, in turn, will cleave and activate the executioners: caspases-3 and caspases-7. This executioner caspase activity effectively kills the cell within minutes, through the parallel cleavage of hundreds of cell components.

Besides cytochrome c, the mitochondria release of a variety of other proteins that promote caspase activity following (MOMP), is a ‘Second Mitochondria-derived Activator of Caspases’ (SMAC), also called ‘Diablo’. In addition, cytochrome c causes the release of ‘Omi’, also called HtrA2, a serine protease enzyme that blocks the endogenous inhibitor of caspase function, an ‘X-linked Inhibitor of Apoptosis Protein’ (XIAP). SMAC and Omi facilitate caspase activity. Importantly, MOMP often leads to cell death irrespective of caspase activity (Tait, Ichim, & Green, 2014). This alternate caspase-independent form of cell death, most likely relates to the extensive nature of MOMP, such that often all cellular mitochondria undergo permeabilization; leading to a progressive and overwhelming loss of mitochondrial function (Lartigue et al., 2009). If is important to note that most stimuli induce apoptosis via the mitochondrial pathways.

Tumor Necrosis Factor (TNF) is a cytokine mainly produced by activated macrophages, and it is the major extrinsic mediator of apoptosis. Most cells in the human body have two receptors for TNF: the TNF-R1, and TNF-R2. The binding of TNF to the TNF-R1 has been shown to initiate a pathway, which leads to caspase activation, via the intermediate membrane proteins, called the ‘TNF Receptor-Associated Death Domain’ (TRADD) or the ‘Fas-Associated Death Domain’ (FADD).

The first apoptosis signal (Fas) also known as Apo-1 or CD95 binds to the Fas ligand (FasL), a transmembrane protein that is part of the TNF family. The interaction between Fas and Fast results in the formation of the ‘Death-Inducing Signaling Complex’ (DISC), which contains the FADD, caspase-8 and caspase-10 (Chou et al., 2015).

Components of the Bcl-2 family: Following TNF-R1 or FADD activation in mammalian cells, apoptosis is determined by the balance between the members of the Bcl-2 family, the proapoptotic members (BAX, BID, BAK, or BAD) and anti-apoptotic members (Bcl-XI and Bcl-X2). If the balance is in favor of proapoptotic homodimers located in the outer-membrane of the mitochondria, the mitochondrial membrane will become permeable and release the caspase activators, such as cytochrome c and SMAC. The control of proapoptotic proteins by non-apoptotic proteins is not completely understood, but in general, proapoptotic members are activated by the BH3 proteins, part of the Bcl-2 family, first identified in B-cell lymphoma 2. (Chou et al., 2015).

(Bcl-2) is encoded in humans by the Bcl-2 gene, which is the founding member of the Bcl-2 family of regulatory proteins. They regulate apoptosis by either inducing (proapoptotic proteins) or inhibiting it (anti-apoptotic proteins). Bcl-2 is specifically considered as an important anti-apoptotic protein and is thus classified as an oncogene. (Williams & Cook, 2015)

Caspases are cysteine-dependent aspartate-specific proteases. There are two types of caspases: initiator caspases and effector caspases. The activation of initiator caspases requires binding by a specific oligomeric activation protein. Effector caspases are then activated by initiator caspases through proteolytic cleavage. The active effector caspases then proteolytically degrade a host of intracellular proteins to carry out the cell death program. Caspases are proteases with a well-defined role in apoptosis, but increasing evidence indicates there are multiple functions for caspases other than apoptosis. Caspase-1 and caspase-11 have roles in mediating inflammatory cell death by pyroptosis (Shalini, Dorstyn, Dawar, & Kumar, 2014).

Caspase-independent apoptic pathway: Apoptosis can also be induced by the caspase-independent pathway called the ‘Apoptosis-Inducing Factor’ (AIF) (Broker, Kruyt, & Giaccone, 2005).

P53 is a tumor-suppressor protein. If accumulates when DNA is damaged by a chain of biochemical factors. Part of this pathway includes alpha-interferon and beta-interferon, which induce transcription of the p53 gene, resulting in the increase of p53 protein level and enhancement of cancer cell apoptosis. First, p53 prevents the cell from replicating by stopping the cell cycle at G1, the interphase, it gives the cell time to repair; however, it will induce apoptosis if damage is extensive and repair efforts fail. Any disruption to the regulation of the p53 or interferon genes will result in impaired apoptosis and the possible formation of tumors (Pflaum, Schlosser, & Müller, 2014). Cell cycle can also be arrested in the G2/M phase by the intrinsic apoptosis proteins, p21 or p27 (Li et al., 2015).

### THE PRIOR ART

Currently, no supplements like the ones described in this patent are on the market.

### SUMMARY OF THE INVENTION

Almost daily, we learn about new benefits from the hunter-gatherer diet. For example, in an examination of genetically similar tribes, with divergent food resources, a marked difference exists in dietary results. The Amazonian Kawymeno Waorani hunter-gatherer's eyes were recently examined. To the doctor's surprise, myopia, or nearsightedness, did not exist in the tribe at any age. However, the neighboring Kichwa agrarian tribe were found to have myopia at the normal rate (London & Beezhold, 2015). The significant divergence between the two tribes is a result of the difference in their diet.

About ten thousand years ago, man started moving from hunter-gatherer food to an agricultural diet. This change in food resulted in increased infant mortality, and the currently discussed agriculture-based diseases.

We believe that agriculture-based diets are missing essential plant ingredients that regulate apoptosis pathways. Also, we consider that many of today's diseases result from a deficiency of apoptosis modulation provided by phytoestrogens. As previously documented, most of the medical and scientific community currently advocate the opposite.

As explained above, both testosterone and estradiol circulate in the bloodstream, bound mostly (60 to 70%) to the sex hormone-binding globulin (SHBG), and to a lesser extent, sex hormones are bound to serum albumin (30 to 40%). Only 1-2% of sex hormones are unbound or free, and only the free unbound form of a hormone can have an effect, and activate its receptor. The bound hormones are protected from being metabolized in the liver until they are needed. Phytoestrogens compete with the sex hormones for their binding sites on SHBG, and albumin, resulting in free, active hormones, which would optimize their respective mate and female characteristics.

More androgens would keep men and women stronger throughout their life, and reduce or prevent post-menopausal problems currently experienced by women. Today, up to 50% of healthy men, between ages 50 to 70, have lower than normal levels of testosterone. The average Western vegetarian consumes 1-3 mg per day of isoflavones, while Asians consume about 50 mg per day. (Bilal et al., 2014). If phytoestrogens are consumed in greater quantities, people would have strong bones Dike those of hunter-gatherers, current hormone replacement therapy could possibly be eliminated.

Furthermore, new science is emerging that indicate how phytoestrogens modulate apoptosis, which repair or eliminate aberrant pre-disease cells. We also believe the absence of the phytoestrogens have contributed to many of the agriculture-based diseases currently facing United States citizens.

Hunter-gatherers learned that certain food choices, and food combinations, made them sick. They observed the food consumed by animals and other people, then made their choices randomly.

We describe supplements that provide healthy structural and functional support for bones, joints, nerves, brain, endocrine and immune systems. Then we targeted ingredients based on the apoptosis pathways they modulate. Otherwise, our supplement ingredient selections would have to be made by trial and error. Our approach tailors the food supplement to the apoptosis required for that system.

### DETAILED DESCRIPTION OF THE INVENTION

A 2013 study, from the Canadian Institutes of Health Research, estimated that there are about 65,000 dietary supplements on the market, consumed by more than 150 million Americans. People have too many choices, and most of these current ingredient choices are not based on good science. Our supplement ingredients are selected based on the apoptosis pathways they modulate.

For example, we choose ingredients that reduce inflammation in bones and joints by modulating the targeted apoptosis pathways. We choose other ingredients to stop defective cells from reproducing, and to help them repair themselves. If they cannot be repaired, the ingredients stimulate apoptosis, and consequently remove defective cells without causing harmful inflammation.

Apoptosis, or programmed cell death, is involved in numerous human conditions including neurodegenerative diseases, ischemic damage, auto-immune disorders and many types of cancer. (Zeng et al., 2015) In these cases, we pick ingredients that stimulate both the intrinsic and extrinsic apoptosis pathways at different sites.

This rational scientific approach enables us to select ingredients that do not duplicate the desired effect, or even worse, cancel out the desired effects. We select ingredients that work at different apoptosis pathway sites and in other selected pathways to achieve the desired outcomes. Otherwise, our selections would be made by trial and error, as in food supplement markets. Supplements can have no effect, or possibly even a negative effect, when selected without an understanding of the apoptosis pathways.

### Seven Characteristics Desired for Apoptosis-Based Daily Supplements

First, at least one of the daily dietary supplements should contain at least two different ingredients that modulate apoptis pathways at different sites or pathways promoting the desired outcome.

It should also be clear that the apoptotic pathways were not originally put here by our creator for us to develop new drugs. The pathways were designed to keep us healthy when we eat the food God provided. We believe that cancer is not always the result of an overgrowth of cells.

Instead, cancer is sometimes caused from a deficiency of apoptosis in aberrant, pre-cancer cells. Apoptosis is generally the safest, and most common method that the body uses to eliminate damaged, precancerous or unneeded cells. When cells cannot be repaired, apoptosis is turned on, and then the cell is safely eliminated. Apoptosis is controlled by cell signals that can originate from inside (intrinsic pathways) or outside (extrinsic pathways) the cell. So, affecting apoptosis is another way food, or food supplements, maintain and support good health.

Second, at least one of the daily dietary supplements should contain the galactins like those found in human milk and plant manna.

Manna exudates from the leaves or branches of plants and frees, and from plant punctures made by insects, or artificial plant incisions. If is usually associated with hot, dry climates of the world, but manna is found all over the globe, including the Americas. European residents, or travelers in the east, adopted Pedro Teixeira's (ca, 1590) description of, “All mana,” as sap or gum from one tree or another is affirmed, and the traditional stories of if coming with dew, are inventions based on bad evidence (Donkin 1980).

Manna is the lifeblood of plants; if contains saccharides, proteins, vitamins and minerals. Several tree gums have been used during famines to sustain life around the world, for months at a time.

Dr. Bill McAnalley, a co-inventor of this patent application, attended an Aboriginal medicinal field trip near Alice Springs, Australia in 2002. During the field trip, he was able to witness acacia trees, with leaves having small holes made by insects. The sap leaked from the holes in the leaves, and quickly dried into very thin threads, six to twenty inches long. It looked and tasted like cotton candy. The aboriginal tour guide said that it was a favorite of the Aboriginal children, and informed Dr. McAnalley that in English their word translates as ‘manna’. The following studies demonstrate some of the health benefits of manna.

African Acacia Senegal manna significantly relieved adenine-induced chronic renal failure in rats. It reduced TNF-α, a pro-inflammatory cytokine. The oxidative stress markers, glutathione and superoxide dismutase, were also significantly reduced (Ali et al., 2013).

A randomized, double-blind study using 427 college students who received either 2.5, or 5.0 grams per day, of a galactan (mannan) supplementation for eight weeks, during the time of their fall final exams. The investigators found that the supplement significantly (P=0.0002) reduced the frequency of acute psychological stress induced gastrointestinal dysfunction and the number of days with a cold or flu (Hughes et al., 2011).

Manna is composed of both, soluble and insoluble galactan fibers. The type of fiber is related to the development of colon cancer. In a population-based fiber study conducted with 231 subjects and 391 controls in Utah between 1979 and 1983, some of the fibers tested, consistently decreased the risk of colon cancer in both males and females. Of the non-cellulose polysaccharides examined, fibers containing mannose and galactose were most protective against cancers in the ascending colon of males, whereas fibers containing galactose and uronic acid were most protective against cancers in the ascending colon in females. A high intake of fruits and vegetables was also associated with the reduction of colon cancer in males and females. High intake of grain cellulose fiber was not protective against colon cancer (Slattery et al., 1988).

All grains are members of the grass family and were not a regular part of the hunter-gatherer's diet. Grasses were not exploited as food until 12,000 years ago, when they were first domesticated and evolved into today's grains (Liu, Bestel, Shi, Song, & Chen, 2013). The hunter-gatherer's biochemistry had not adjusted to using cellulose fiber from grains.

Grains did enable people for the first time to feed and maintain animals, which provided a regular supply of red meat and milk. Most people are aware of the health problems associated with lactose intolerance, and too much red meat.

Today, approximately 70% of the adult human population world-wide is lactose intolerant (Hansen et al., 2015). This demonstrates how long it fakes for mankind to genetically adapt to new foods, like milk and grains. This also explains why grain fiber is not protective against cancers in the ascending colon.

Mother's milk contains everything a baby human needs to grow and be healthy. Mother's first milk, colostrum, contains IgA antibodies made by the mother to protect her baby against local pathogens when first born. A newborn baby's gastrointestinal tract is sterile; when bacteria is first introduced from their environment and/or the mother's skin, as babies start to nurse, lactoferrin is the major protein in milk, and it acts as an anti-inflammatory by turning off these inflammatory cytokines: IL-1, IL-6 and TNF-α. These cytokines are produced when the baby's gut begins to grow newly introduced bacteria, probiotics. This helps to explain why breast-fed babies lose significantly less weight than bottle-fed babies during the first week of feeding. Inflammatory responses burn more calories resulting in weight loss (Ben et al., 2008).

Lactose is the major sugar in milk, a disaccharide composed of galactose and glucose. Human milk also contains a high concentration of galacto-oligosaccharides, however, cow's milk contains only trace amounts of these oligosaccharides (Hanson, Korotkova, & Telemo, 2003). Galacto-oligosaccharides are small galactans, only recently added to baby milk formulas to stimulate the growth of intestinal Bifidobacteria and Lactobacilli (Ben et al., 2008). Mother's milk provides small galactans to feed and maintain a healthy live culture of probiotics, which are required for the baby to successfully digest food. This is necessary for the baby to shift, when moving from mother's milk to hunter-gatherer food. Then galactan fibers in the hunter-gatherer's food, like manna, fake over the job of feeding the required probiotics.

The protective effect of fruit and vegetable fibers appear to be related to the galactose content (Evans et al., 2002). This provides further evidence for the association between diet fiber types and colon cancer. If appears to be caused mainly by a deficiency of galactans.

Third, most current supplements require too many unwanted ingredients. Dietary supplements are best when made without flowing agents, binding agents, or fillers needed for modern drug manufacturing processes. This will insure better compliance by requiring fewer and smaller capsules per daily dose.

Current automatic encapsulating and tablet machines, initially designed for the drug industry, and require standard flowing, filling and binding agents. If these machines were used, our products would contain from 5% to 20% of the desired supplement ingredients. This requires the consumer to take as many as 20 capsules, or tablets, to get the same amount of the desired supplement ingredients from 1 capsule filled manually.

A recent investigation, reported by CBS News 2/11/15, 11:15 AM, led by New York Attorney General Eric Schneiderman, focused on a variety of herbal supplements from four major retailers: GNC, Target, Walmart and Walgreens. Lab tests determined that only 21 percent of the products actually had DNA from the plants advertised on the labels. Some of the products only contained filler. The retailer with the poorest showing was Walmart, where only 4 percent of the products tested showed DNA from the plants listed on the labels.

“This investigation makes one thing abundantly clear: The old adage ‘buyer beware’ may be especially true for consumers of herbal supplements,” Schneiderman said. His office issued cease and desist letters to the retailers felling them to stop sales of the products.

The automated tableting and capsuling machines were designed to be used by the drug industry, not the supplement industry. For example, a 5 milligram prednisone drug tablet may weight 250 milligrams, giving a tablet with 2% active ingredient and 98% flowing, filling and binding agents used in tableting production. Automated machines work well with highly potent drugs that require small amounts of active ingredients to be effective. A manual manufacturing process must be used to allow the most parsimonious method for supplemental products.

Fourth, daily dietary supplements should contain at least one ingredient that is an adaptogen.

Adaptogens are supplements that nourish the whole body and support a variety of cellular structures and functions. Their nourishment enables the body to more effectively deal with both emotional and physical stresses. Drugs are designed to target a specific area of physiology.

For example, the Russians developed a supplement largely made from Siberian ginseng, an adaptogen, which enhanced their Olympic athlete's performance. (Personal communication with a Russian scientist).

Fifth, a daily dietary supplement should contain the DGAC shortfall nutrients in food form.

Hunter-gatherer foods contain the DGAC short-fall nutrients in food form: vitamin A, vitamin D, vitamin E, vitamin C, folate, calcium, magnesium, fiber, and potassium. For adolescent and premenopausal females, iron is also a shortfall nutrient.

Sixth, at least one dietary supplement should be specifically designed to stimulate the immune system at many areas needed. The immune system supplements can be taken every day, but they are most effective if taken before going into the places where sick people are concentrated. Taking 2-3 doses immediately before or after exposure to sick people, when traveling, shopping, going to hospitals, games or churches, etc. is the best protocol.

As previously documented, intrinsic and extrinsic apoptosis pathways may prevent viral, and bacterial infection.

Seventh, every dietary supplement should be chemically tested to assure consistency, batch to batch.

Fourier transform infrared (FT-IR) spectroscopy is based on the principle that molecules can absorb certain wavelengths in the electromagnetic spectrum. This absorption can be attributed to the different bond groups, or functional groups, that are contained in the molecule. The intensity, shape and position of the peaks in the spectrum give the quality details for the sample being analyzed.

Many industries use FT-IR spectroscopy to analyze chemicals or components both to study chemical structures, and in many industries, as a screening tool to make sure a chemical, or component, is in fact what it is supposed to be. For example, in the dietary supplement industry FT-IR spectroscopy can be used to compare the chemical spectrum of an ingredient or component to a library of known ingredients. With modern computer programs, the computer can compare the spectra, and give within a degree of certainty that an ingredient or component is the same as the desired known ingredient.

As should be expected, there will be some variation from one sample of a natural or whole food ingredient to another. This is due to variations from one plant to the next, from one growing region to the next and just as relevant, from one year to the next. Expecting the spectra to match 100% with the library, or an ingredient standard, would be absurd.

However, one could utilize FT-IR spectroscopy to make sure that there is an adequate or desired amount of a certain functional or bond group within
a sample
a mixture of different ingredients, or
a mixture of the same ingredient from different suppliers or batches from the same supplier.

Using the same principle one could utilize FT-IR spectroscopy to get a desired level of functional groups by
mixing different ingredients or samples,
mixing different mixtures,
fortifying a sample or ingredient that is “the same” from one supply with another
fortifying a mixture with an ingredient or sample from a different supply,
fortifying a mixture with another mixture

This must not be obvious to the supplement industry, or many retailers like, GNC, Target, Walmart and Walgreens would not have been selling fraudulent supplements.

### CONCLUSION

Scientists ail over the world are using apoptosis screens to identify single ingredients that can be chemically modified into new chemical entities for specific drug uses. Alternatively, we use the apoptosis screens to identify pre-agriculture plants, mannas, fruits, vegetables, roots, leaves and stems that effect the specific desired apoptosis pathways. Then we combined these ingredients to make supplement compositions composed of many naturally occurring ingredients.

The compositions of the ingredients are selected based on targeted apoptosis effects that should reduce, and hopefully, eliminate some of the current agriculture-based diseases. We have explained why phytoestrogens are not a problem; instead their deficiency may ultimately be responsible for many common current health problems.

Using the apoptosis pathways studies, we can provide supplements that support the structure and function for the endocrine and immune system, bones, joints, nerves, and the brain. The ingredients are selected based on the apoptosis pathways that they modulate.

What is an effective amount of our dietary supplements? In theory any amount of a dietary supplement ingredient can have some effect. FDA has established a Daily Value (DV) in their food labeling guide (21CFR 101.9(c)) for 32 nutrients in foods. 2% of the DV is the FDA's label guide for ingredient amounts that can be effective when added to their diet.

Daily Values are based on the RDAs for fats, carbohydrates, proteins, fiber, vitamins and minerals. However, RDAs have not been established for herbs, fruits, roots, spices and other botanicals.

Ingredients contain 80% to 90% water, some, like Aloe vera gel contain 99.5% water. Our supplements must be free of moisture (for example freeze-dried) to be stable until consumed. Freeze-dried foods, stored without oxygen, can last up to 30 years.

One ounce (wt.) of a fresh hydrated ingredient is 28.35 grams. Assuming an average of 90% water, then the weight of one ounce dehydrated is 2.835 grams. This amount of dried (manna, roots, fruits or herbs) has been shown to be effective by itself.

For Example, in a randomized, double-blind study using 427 college students who received either 2.5, or 5.0 grams per day, of a galactan (mannan) supplementation for eight weeks, during the time of their fall final exams, the investigators found that the supplement significantly (P=0.0002) reduced the frequency of acute psychological stress induced gastrointestinal dysfunction and the number of days with a cold or flu (Hughes et al., 2011).

We have found that an immediate effective amount of dried ingredients such as manna, roots, fruits or herbs individually or in combination is 2.5 grams. Our supplements contain 625-750 milligrams per capsule. Therefore 4 capsules taken at once or two capsules in the morning and two in the evening is 2.5 to 3 grams, an effective dose in all subjects we have tested.

Many of our test subjects have found that one capsule containing 500 milligrams per day of the compositions in examples 1-8 described below is an effective maintenance dose. Using 500 milligrams as the DV, 2% of the DV is 10 milligrams an amount that may have some effect. 10 milligrams is a tot considering that many cellular apoptosis molecules are measured in picograms.

### DEFINITIONS OF TERMS USED IN THIS PATENT

Adaptogens are supplements that nourish the whole body and support a variety of cellular structures and functions. Their nourishment enables the body to more effectively deal with both emotional and physical stresses.

Apoptosis is also known as programmed cell death. This is nature's way of eliminating unhealthy cells that no longer function properly and cannot repair themselves. The average human body replaces an estimated one million cells per second or an average two billion defective cells daily by apoptosis. Pre-agricultural foods provided the nutrients the body needs to effectively use its apoptosis pathways and be healthy.

Effective amounts are as little as 10 milligrams per day. We prefer to give 2-3 grams per day of each supplement in examples 1-8 described below for a month as a loading dose, to achieve an effect, then let the individual gradually adjust the amount until they find their individual effective amount, (see explanation above for more detail)

Food form nutrients are not chemically synthesized, or isolated, from a plant but are contained in the natural dried (preferably freeze-dried) plant matrix.

Immune system modulators are ingredients that can adjust the immune response, to a desired level, by immuno-potentiation or immuno-suppression and can induce immunological tolerance.

Ingredients include hunter-gatherer foods, like manna or genetically unmodified pre-agricultural foods, especially roots and tubers, herbs, fruits, vegetables or spices from plants, or plant parts.

Manna is a common food of Hunter-gatherers, the exudates from the leaves or branches of shrubs, or trees, or from punctures by insects, or artificial incisions in those plants. When pollen is in short supply, it is often used by bees to make honey.

### EXAMPLES OF COMPOSITIONS

Use FT-IR spectroscopy on all of the dietary supplement components, and final compositions, to assure supplement consistency from batch to batch. All supplement compositions will be supplied in capsules, or as bulk powder, without any flowing agents, binding agents or fillers”.

### Example 1

### A Dietary Supplement Composed of these Ingredients

The following studies were used to select two ingredients to support apoptosis at different sites and one manna ingredient to provide galactans for colon health.

250 milligrams of Trichosanthes kirilowii recently shown to significantly induced G2-M arrest, and apoptosis in non-small cell lung cancer cell growth (Ni et al., 2015). A Trichosanthes kirilowii ethanol extract reduced cisplatin-induced acute renal failure by increasing anti-oxidative enzyme levels, decreased lipid peroxidation levels and reduced histopathological alterations in the kidney with decreased apoptotic cells (Seo et al., 2015). A polysaccharide of Trichosanthes kirilowii can induce the apoptosis of MCF-7 estrogen (+) breast cancer cells, by the activation of intracellular Caspase-3 and Caspase-8 (Cao, Xu, Xu, Jin, & Shen, 2012).

250 milligrams of Dioscorea opposita, Chinese yam, that contains components that promoted the proliferation of human endometrial epithelial cells by up regulating Bcl-2 and down regulating the Bax/Bcl-2 ratio (Ju, Xue, Huang, Zhai, & Wang, 2014).

250 milligrams of Brazilian Acacia mearnsii gum, a manna, that contains galactans with approximately 40%—galactose, 30%—arabinose, 17%—uronic acids, 10%—rhamnose with a trace of glucose (Grein et al., 2013). Put the ingredients in a capsule without standard flowing agents, binding agents or fillers and take four of these capsules a day for a dose of 3 grams.

### Example 2

### A Dietary Supplement Comprised of these Ingredients

The following studies were used to select three ingredients to support apoptosis at different sites and one manna ingredient to provide galactans for colon heath.

200 milligrams of Trichosanthes kirilowii recently shown to significantly induced G2-M arrest, and apoptosis in non-small cell lung cancer cell growth (Ni et al., 2015). A Trichosanthes kirilowii ethanol extract reduced cisplatin-induced acute renal failure by increasing anti-oxidative enzyme levels, decreased lipid peroxidation levels and reduced histopathological alterations in the kidney with decreased apoptotic cells (Seo et al., 2015). A polysaccharide of Trichosanthes kirilowii can induce the apoptosis of MCF-7 estrogen (+) breast cancer cells, by the activation of intracellular Caspase-3 and Caspase-8 (Cao, Xu, Xu, Jin, & Shen, 2012).

200 milligrams of Dioscorea opposita, Chinese yam, that contains components that promoted the proliferation of human endometrial epithelial cells by up regulating Bcl-2 and down regulating the Bax/Bcl-2 ratio (Ju, Xue, Huang, Zhai, & Wang, 2014).

100 milligrams of Curcuma tonga, turmeric roof, powder that provides apoptosis support at these sites, NF-κB, p38 and p53. (Jiang, Jiang, Li, & Zheng, 2015).

250 milligrams of Brazilian Acacia mearnsii gum tree that contains a galactan with approximately 40%—galactose, 30%—arabinose, 17%—uronic acids, 10%—rhamnose with a trace of glucose (Grein et al., 2013). Put the ingredients in a capsule without standard flowing agents, binding agents or fillers and fake four of these capsules a day for a dose of 3 grams.

### Example 3

### A Dietary Supplement Comprised of these Ingredients

The following studies were used to select three ingredients to support apoptosis at different sites, one manna ingredient to provide galactans for colon heath, one adaptogen, to nourish the whole body and support a variety of cellular structures and functions.

150 milligrams of Trichosanthes kirilowii recently shown to significantly induced G2-M arrest, and apoptosis in non-small cell lung cancer cell growth (Ni et al., 2015). A Trichosanthes kirilowii ethanol extract reduced cisplatin-induced acute renal failure by increasing anti-oxidative enzyme levels, decreased lipid peroxidation levels and reduced histopathological alterations in the kidney with decreased apoptotic cells (Seo et al., 2015). A polysaccharide of Trichosanthes kirilowii can induce the apoptosis of MCF-7 estrogen (+) breast cancer cells, by the activation of intracellular Caspase-3 and Caspase-8 (Cao, Xu, Xu, Jin, & Shen, 2012).

150 milligrams of Dioscorea opposita, Chinese yam, that contains components that promoted the proliferation of human endometrial epithelial cells by up regulating Bcl-2 and down regulating the Bax/Bcl-2 ratio (Ju, Xue, Huang, Zhai, & Wang, 2014).

150 milligrams of Curcuma tonga, turmeric root, powder that provides apoptosis support at these sites, NF-κB, p38 and p53. (Jiang, Jiang, Li, & Zheng, 2015).

150 milligrams of Brazilian Acacia mearnsii gum tree that contains a galactan with approximately 40%—galactose, 30%—arabinose, 17%—uronic acids, 10%—rhamnose with a trace of glucose (Grein et al., 2013).

150 milligrams of Siberian ginseng, Eieutherococcus senticosus, or golden root, Rhodiola rosea, to the dietary supplement composition in example 2 above. Both, are fully compliant with the definition of an adaptogen (Panossian & Wagner, 2005). Put the ingredients in a capsule without standard flowing agents, binding agents or fillers and take four of these capsules a day for a dose of 3 grams.

### Example 4

### A Dietary Supplement Comprised of these Ingredients

The following studies were used to select three ingredients to support apoptosis at different sites, one manna ingredient to provide galactans for colon heath, one adaptogen, to nourish the whole body and support a variety of cellular structures and functions, and one ingredient to supply DGAC shortfall nutrients.

100 milligrams of Trichosanthes kirilowii recently shown to significantly induced G2-M arrest, and apoptosis in non-small cell lung cancer cell growth (Ni et al., 2015). A Trichosanthes kirilowii ethanol extract reduced cisplatin-induced acute renal failure by increasing anti-oxidative enzyme levels, decreased lipid peroxidation levels and reduced histopathological alterations in the kidney with decreased apoptotic cells (Seo et al., 2015). A polysaccharide of Trichosanthes kirilowii can induce the apoptosis of MCF-7 estrogen (+) breast cancer cells, by the activation of intracellular Caspase-3 and Caspase-8 (Cao, Xu, Xu, Jin, & Shen, 2012).

100 milligrams of Dioscorea opposita, Chinese yam, that contains components that promoted the proliferation of human endometrial epithelial cells by up regulating Bcl-2 and down regulating the Bax/Bcl-2 ratio (Ju, Xue, Huang, Zhai, & Wang, 2014).

100 milligrams of Brazilian Acacia mearnsii gum tree that contains a galactan with approximately 40%—galactose, 30%—arabinose, 17%—uronic acids, 10%—rhamnose with a trace of glucose (Grein et al., 2013).

100 milligrams of Curcuma tonga, turmeric roof, powder that provides apoptosis support at these sites, NF-k B, p38 and p53. (Jiang, Jiang, Li, & Zheng, 2015).

100 milligrams of Siberian ginseng, Eleutherococcus senticosus, or golden roof, Rhodiola rosea, Both, are fully compliant with the definition of an adaptogen (Panossian & Wagner, 2005). Put the ingredients in capsules containing 750 mg each without standard flowing agents, binding agents or fillers and fake four capsules daily for a daily dose of 3 grams.

250 milligrams of Baobab fruit, Adansonia digitata L, Adansonia digitata L. is known as the ‘tree of life’ and ‘the king of fruits’. It is rich in antioxidants, amino acids, vitamins A, B1, B2, B3, B6, C, Magnesium, Calcium, Potassium, Manganese, Zinc, Phosphorus, Iron, protein and dietary fiber (soluble and insoluble). Baobab fruit contains six times the vitamin C found in oranges, three times the iron found in spinach, three times the antioxidants found in blueberries, three times the calcium found in milk, and six times the potassium of bananas. Freeze-dried powder contains food form DGAC shortfall nutrients. Put the ingredients in a capsule without standard flowing agents, binding agents or fillers and take four of these capsules a day for a dose of 3 grams.

### Example 5

### A Dietary Supplement Comprised of these Ingredients

The following apoptosis studies were used to select ingredients to support bones, joints and the endocrine system. Osteoarthritis is characterized by a loss of articular cartilage, accompanied by inflammation, and if is the most common age-associated degenerative disease.

250 milligrams of Dioscorea villarosa, American yam, induced G2/M cell cycle arrest and activates apoptosis by inhibiting the expression of p21 and p27 (Li et al., 2015). If has been used for hundreds of years to treat rheumatism and arthritis-like ailments. It contains thirteen percent phytoestrogens that support the endocrine system, as explained by the relative binding affinities (RBAs) of the various hormones discussed above.

250 milligrams of Emblica officinalis, Amalaki, increased the expression levels of Fas, a critical member of the apoptotic pathway, which may be a treatment of rheumatoid arthritis and osteoporosis, by activating programmed cell death of human primary osteoclasts that cause both diseases. (Penolazzi et al., 2008).

250 milligrams of Coleus forskohliiis, coleus, that naturally reduces inflammation and significantly decreases the expressions of Bcl-2, and Bcl-x (Sun et al., 2011). Put the ingredients in a capsule without standard flowing agents, binding agents or fillers and take four of these capsules a day for a dose of 3 grams.

### Example 6

### A Dietary Supplement Comprised of these Ingredients

The following apoptosis studies were used to select ingredients to support the brain and nerves.

250 milligrams of Withania somnifera, ashwagangha, reduces Parkinson symptoms by reducing Bax and inducing Bcl-2 expression, resulting in the reduced expression of the pro-inflammatory markers of astrocyte activation (Prakash et al., 2014).

250 milligrams of Astragalus membranaceus, milk vetch roof, contains cycloartane triterpene saponin, a phytoestrogen that blocks procaspase-8, resulting in the inhibition of caspase-3 and procaspase-9 activities. These changes are accompanied with down-regulation of Bax and p53, and up-regulation of Bcl-2 and Bcl-xL. If is also an adaptogen that helps the body deal with various stresses, including physical, mental, or emotional stress (Kim, Kim, & Yang, 2014).

250 milligrams of Bacopa monnieri, brahmi, reduces chronic systemic brain inflammation by down-regulation of NO and TNF-α (Williams, Munch, Gyengesi, & Bennett, 2014). If supports both short- and long-term memory function as well as possibly enhancing learning, and concentration by nourishing the nervous system. It is a neuroprotective agent for the prevention of cognitive deficits in schizophrenia (Piyabhan & Wetchateng, 2014). Therefore, treating patients with Brahmi extract may be an alternative direction for ameliorating neurodegenerative disorders associated with the overwhelming oxidative stress, such as Alzheimer's disease (Limpeanchob, Jaipan, Rattanakaruna, Phrompittayarat, & Ingkaninan, 2008). Put the ingredients in a capsule without standard flowing agents, binding agents or fillers and take four of these capsules a day for a dose of 3 grams.

### Example 7

### A Dietary Supplement Comprised of these Ingredients

The following apoptosis studies were used to select ingredients that support apoptosis and the immune system at different sites

250 milligrams of Ganoderma lucidum, reishi mushroom, is known as an adaptogen and an immune system modulator. A polysaccharide obtained from Ganoderma lucidum suppressed HL-60 acute myeloid leukemia cells by activating the p38 and JNK MARK, part of the intrinsic apoptosis pathways (Yang, Yang, Zhuang, Qian, & Shen, 2014).

250 milligrams of Dioscorea villarosa, American yam, induces apoptosis by activating caspase-3, caspase-8 and caspase-9, part of the intrinsic apoptosis pathways (Li et al., 2015).

250 milligrams of Lentinus edodes, shiitake mushroom, activates caspase-3 and caspase-8 in the death receptor intrinsic pathway responsible for the apoptotic death of liver cancer, HepG2, cells (Yukawa, Ishikawa, Kawanishi, Tamesada, & Tomi, 2012). Put the ingredients in a capsule without standard flowing agents, binding agents or fillers and take four of these capsules a day for a dose of 3 grams.

### Example 8

### A Dietary Supplement Comprised of these Ingredients

The following studies were used to select ingredients to provide galactans (manna) to feed the colon and protect against colon cancer (Slattery et al., 1988) and the frequency of acute psychological stress induced gastrointestinal dysfunction and the number of days with a cold or flu (Hughes et al., 2011).

1500 milligrams of Larix sibirica, larch tree, arabinogalactan manna can stimulate natural killer (NK) cell cytotoxicity, part of the extrinsic apoptosis pathways (Kelly 1999).

1500 milligrams of Brazilian Acacia mearnsii gum tree that contains a galactan with approximately 40%—galactose, 30%—arabinose, 17%—uronic acids, 10%—rhamnose with a trace of glucose (Grein et al., 2013). Put 100 grams of the ingredients on a jar without standard flowing agents, binding agents or fillers and take a daily dose of 3 grams per day in yogurt, juice or some other food.

### Example 9

### A Daily Dietary Supplement Combination Composed of

Two capsules from each of Examples 5, 6, 7 and 3 grams of example 8 for a total of 7.5 grams of freeze-dried ingredients. This is 750 times the minimum effective amount of 10 milligrams explained above.

What is claimed is:

1. A apoptotic dietary supplement composition based on the apoptosis pathways effected, comprising an apoptotic effective amount of at least two different hunter-gatherers' foods that modulate apoptosis pathways at different sites, and comprises at least one galactan containing galactose fibers and further comprising no flowing agents, binding agents, or fillers.

2. A dietary supplement composition according to claim 1, wherein at least three different hunter-gatherers' foods that modulate the apoptosis pathways at different sites, and comprises at least one galactan containing galactose fibers, and comprising no standard flowing agents, binding agents or fillers.

3. A dietary supplement composition according to claim 2, wherein said composition further comprises at least one other ingredient considered an adaptogen.

4. A dietary supplement composition according to claim 2, wherein said composition further comprises at least one other hunter-gatherer ingredient, further comprising at least one from the United States, 2015 Dietary Guidelines Advisory Committee (DGAC) short-fall nutrients in food form.

5. A dietary supplement composition according to claims 1 through 4, that is chemically tested to assure composition consistency from batch to batch.

6. A dietary supplement composition according to claim 5, that also supports bones, joints, and the endocrine system.

7. A dietary supplement composition according to claim 5, that also supports the brain and nerves.

8. A dietary supplement composition according to claim 5, that also supports the immune system and apoptosis.
